(19) United States
(12) Patent Application Publication
(10) Pub. No.: US 2024/0156731 A1
(43) Pub. Date: May 16, 2024

(54) AN OLEOGEL COMPOSITION COMPRISING AN ETHYLCELLULOSE AND AN OILY ACTIVE INGREDIENT

(71) Applicant: NUTRITION & BIOSCIENCES USA 1, LLC, Rochester, NY (US)

(72) Inventors: Rebecca Putans, Midland, MI (US); True Rogers, Midland, MI (US); Gerald Schalau, Midland, MI (US); Kevin O'Donnell, Midland, MI (US)

(21) Appl. No.: 18/549,612

(22) PCT Filed: Mar. 8, 2022
(86) PCT No.: PCT/EP2022/055894

§ 371 Date: Sep. 8, 2023

Related U.S. Application Data
(60) Provisional application No. 63/158,590, filed on Mar. 9, 2021.

(51) Int. Cl. A61K 9/06; A61K 47/02; A61K 47/38; A61K 47/44

(52) U.S. Cl. CPC A61K 9/06; A61K 47/02; A61K 47/38; A61K 47/44

(57) ABSTRACT

An oleogel composition comprises an ethylcellulose polymer, a first, non-volatile oil which is an active ingredient and a second oil selected from the group consisting of triglyceride oils and mineral oils.

### FIELD OF INVENTION

The present invention relates to an oleogel composition comprising an ethylcellulose and a non-volatile oil which is an active ingredient and a unit dosage form comprising said oleogel composition.

### BACKGROUND OF THE INVENTION

A critical challenge in the pharmaceutical industry is the large percentage of active ingredients that are poorly water-soluble. Many of these are oily or lipophilic in nature which makes conventional dosage forms such as tablets difficult to produce. One strategy for pharmaceutical companies to explore is reformulation or different delivery methods of existing active ingredients to improve their solubility and bioavailability. These active ingredients are typically formulated into an immediate release dosage form. The drug will commonly be dissolved in an oil or lipid solution and filled into a soft-shelled capsule. For particularly difficult active ingredients self-emulsifying drug delivery systems (SEDDS) can be used. These formulations, however, can become rather complicated with the addition of solvents, co-solvents, surfactants etc.

C. M. O'Sullivan et al., Food and Function 8, 2017, pp. 1438-1451, describe ethylcellulose oleogels made with canola oil and containing β-carotene as a lipophilic bioactive agent. Oleogels were formed with 10% ethylcellulose with a viscosity of 10 cP, 20 cP or 45 cP. The greatest hardness of the oleogels was achieved with 45 cP ethylcellulose. It was found that β-carotene release from the oleogel containing 45 cP ethylcellulose was significantly slower than from oleogels containing 10 cP and 20 cP ethylcellulose.

Liquid and oily active ingredients are delivered today either through a hard or soft shell capsule or are formulated as a liquid product such as a suspension. These delivery methods often result in immediate release offerings and it may be difficult to provide adequate drug loading. Oral solid dosage forms are often preferred by customers, but liquid and oily active ingredients are difficult to formulate into a solid. Drug formulators also prefer to work with solid materials, as liquid handing can be challenging especially with viscous, sticky, resinous oily active ingredients. Structuring of oily active ingredients offers a solution to create a solid form of oily active ingredients that can be used as-is or further processed to create a more typical solid oral dosage form (for example capsule or tablet).

Oleogels that are structured with ethylcellulose have previously been proposed as a replacement of saturated solid fats in various food products. Thus, WO 2010/143066 describes oleogels that contain ethylcellulose polymer, oil, and surfactant, normally a nonionic surfactant. The addition of a surfactant is indicated to plasticize the ethylcellulose polymer, slow down the gelation process and induce formation of stable, translucent and elastic gels.

S. Yogev and B. Mizrahi, ACS Applied Polymer Materials, 17 Apr. 2020, describe an oleogel delivery system composed of volatile oils structured with ethylcellulose at concentrations between 25 and 50% by weight. The volatile oils (linalool, citrol and Mentha arvensis) are shown to be released in a sustained manner from the oleogels. The oleogels are proposed for topical application to treat fungal infections such as tinea pedis (athlete's foot) and onychomycosis as the structured volatile oils were shown to affect the pathogenic agent (T. rubrum) due to the gradual evaporation of the volatile oil from the oleogel.

### SUMMARY OF THE INVENTION

Structured oil oleogels made with ethylcellulose are proposed by the present inventors as an alternative vehicle for oral delivery of oily, lipophilic active ingredients where the active ingredient is in the form of an oil which is either directly structured by ethylcellulose or, if the oily active ingredient is not miscible with ethylcellulose, it is formulated with another oil that is miscible with the ethylcellulose and/or the oily active ingredient and acts as a carrier for the active ingredient by forming a continuous phase in the gel network created by the ethylcellulose to create a solid gel. The oily active ingredient will slowly diffuse out of the gel network on oral administration.

In a first aspect, the present invention relates to an oleogel composition comprising an ethylcellulose polymer, a first, non-volatile oil which is an active ingredient and a second oil selected from the group consisting of triglyceride oils and mineral oils.

The present composition differs from other lipid-based delivery systems by creating a solid or semi-solid material from a liquid, which may be more easily formulated as unit dosage forms that are more convenient for the patients to take and may even provide sustained release of the active ingredient. The present composition also has the advantage of including only a few components unlike the self-emulsifying drug delivery systems which typically contain solvents, co-solvents and surfactants.

Thus, in another aspect, the invention relates to a unit dosage form comprising said oleogel composition.

### BRIEF DESCRIPTION OF THE DRAWINGS

FIG. 1 is a graph showing the time to flatten a foam layer of a surfactant in the presence of neat simethicone and two oleogel compositions comprising simethicone and sunflower oil in a 40:60 ratio as well as 17% by weight of ethylcellulose of two different viscosities.

FIG. 2 is a graph showing the time to flatten a foam layer of a surfactant in the presence of an oleogel composition comprising simethicone and sunflower oil in a 40:60 ratio as well as 17% by weight of ethylcellulose when the same oleogel composition is placed sequentially in two foamed surfactant solutions in separate jars.

FIG. 3 is a series of photographs showing the stability (shape retention) of ethylcellulose oleogel comprising cannabinoid oil and sesame oil in the ratios 90:10 (top left), 80:20 (top middle), 70:30 (top right), 60:40 (bottom left) and 50:50 (bottom right) after one week of storage at room temperature.

### DETAILED DESCRIPTION OF THE INVENTION

### Definitions

Ethylcellulose polymer, as used herein, means a derivative of cellulose in which some of the hydroxyl groups on the repeating glucose units are substituted with ethyl ether groups. The number of ethyl ether groups can vary. The number of ethyl ether groups is characterized by the “percent ethoxyl substitution.” The percent ethoxyl substitution is based on the weight of the substituted product and determined according to a Zeisel gas chromatographic technique as described in ASTM D4794-94 (2003). The ethoxyl substitution (also called “ethyl ether content”) is from 10 to 55%.

As used herein, the viscosity of an ethylcellulose polymer is the viscosity of a 5% by weight solution of that ethylcellulose polymer in a solvent, based on the weight of the solution. The solvent is a mixture of 80% toluene and 20% ethanol by weight. The viscosity of the solution is measured at 25° C. in an Ubbelohde viscometer.

As used herein, an oil is a material that is liquid at temperatures of 35° C. or less. An oil has no chemical group of the structure -(—CH₂CH₂—O—)ₙ— where n is 2 or more. One category of oils is triglycerides, which are triesters of fatty acids with glycerol. Vegetable oils are triglycerides that may be saturated or unsaturated, i.e. contain one or more double bonds.

As used herein, the term “oleogel” refers to a mixture that contains one or more oils and one or more ethylcellulose polymers forming a network throughout the oil continuous phase; the oleogel is solid at 25° C. The oleogel may be a relatively hard solid or a relatively soft solid.

Any ethylcellulose polymer may be used in the composition of the present invention. The ethoxyl substitution content of the ethylcellulose polymer is preferably 10% or more; more preferably 30% or more; more preferably 40% or more; preferably 45% or more; more preferably 48% or more. The ethoxyl substitution content of the ethylcellulose polymer is preferably 55% or less; more preferably 53% or less; more preferably 52% or less; more preferably 51% or less; more preferably 50% or less.

The ethylcellulose polymer preferably has a viscosity of 5-60 mPa·s when determined as a 5% solution in 80% by weight toluene and 20% by weight ethanol solvent at 25° C. in an Ubbelohde viscometer. The ethylcellulose polymer more preferably has a viscosity of 5-8 mPa·s, 9-11 mPa·s, 18-22 mPa·s or 41-49 mPa·s mPa·s when determined by this method.

Commercially available grades of ethylcellulose polymer which may be used in the invention include, for example, those available under the name ETHOCEL™, from DuPont, including, for example, ETHOCEL™ Standard 7, ETHOCEL™ Standard 10, ETHOCEL™ Standard 20 or ETHOCEL™ Standard 45 with ethoxyl substitution content from 48.0 to 49.5%. Other commercially available ethylcellulose polymers useful in embodiments of the invention include certain grades of AQUALON™ ETHYLCELLULOSE, available from Ashland, Inc., and certain grades of ASHACEL™ ethylcellulose polymers, available from Asha Cellulose Pvt. Ltd.

Preferably the amount of ethylcellulose polymer in the composition is, by weight based on the weight of the composition, 9% or more; more preferably 10% or more; more preferably 12% or more. Preferably the amount of ethylcellulose polymer in the composition is, by weight based on the weight of the oleogel, 20% or less; more preferably 18% or less; more preferably 17% or less.

The oleogel contains a second oil which is a non-volatile oil selected from the group consisting of vegetable oils, animal oils (such as fish oil), mineral oils (such as paraffin oil) or synthetic oils (such as caprylic/capric triglycerides). Preferred oils are unsaturated triglycerides, preferably vegetable oils such as sunflower oil, rapeseed oil (also known as canola oil), grape kernel oil, corn oil, soybean oil, olive oil, flaxseed oil, safflower oil, peanut oil, sesame oil, argan oil, rice bran oil, cottonseed oil, linseed oil, almond oil, coconut oil and palm oil, and mixtures thereof. Preferred vegetable oils have not been hydrogenated or modified by any other chemical reaction.

Preferably the amount of the second oil in the composition is, by weight based on the total weight of the composition, 45-60%.

The oleogel composition may contain a plasticizer selected from the group consisting of stearic acid, triacetin, oleic acid, glyceryl monostearate, glycerol, propylene glycol or polyethylene glycol. It has been found that adding a plasticizer contributes to a higher viscosity of the ethylcellulose oleogel and limits the amount of oil weeping from the oleogel composition.

The oleogel composition may also comprise one or more surfactants to emulsify ethylcellulose in the first, non-volatile oil or the second oil. Examples of suitable surfactants are selected from the group consisting of silicone surfactants and glyceryl monostearate.

The present composition may be made by any of a variety of processes whereby the first, non-volatile oil, the second oil, the ethylcellulose polymer and optional additional ingredients are brought together to form a mixture. Preferably the mixture is heated to a temperature at which the ethylcellulose polymer melts in the second oil such as a temperature of from 130° C. to 160° C. Preferably the mixture is subjected to mechanical agitation in a mixer provided with a stirring blade set to a speed of 400-500 rpm. Preferably the mixture is heated and agitated at the same time. Typical heating times (holding times) range from 10 to 80 minutes, preferably from 20 to 70 minutes, until an oleogel is formed. It has been found that the holding times differ depending on the first, non-volatile oil and/or second oil included in the oleogel composition. Thus, in a preferred embodiment, the mixture is heated and agitated at a temperature of about 150-160° C. for 30 to 40 minutes (e.g. when the first, non-volatile oil is cannabinoid oil) or for 60 to 70 minutes (e.g. when the first, non-volatile oil is simethicone).

When the mixture is heated above the melting temperature of the ethylcellulose, the ethylcellulose is solubilized in the oil(s) to create a three-dimensional gel network upon cooling. Due to the restricted mobility and migration of the oil(s) inside the polymer network, the present oleogels provide the solid-like properties of crystalline triglycerides.

The molten oleogel may then be molded in the desired shape by pouring it into molds followed by cooling to form the hardened dosage form such as a gummy or jelly. The dosage form is preferably cooled to a temperature of less than 30° C., more preferably a temperature of equal to or less than 25° C. or even a temperature equal to or less than 20° C. Alternatively, the molten oleogel may be filled into hard capsules (e.g. HPMC or gelatin capsules) and left to harden with cooling before capping.

It may be advantageous that heating and agitating the mixture is conducted under an inert atmosphere such as under an inert gas atmosphere or in ambient air under vacuum (reduced pressure). Examples of inert gases that may be used according to the present invention include nitrogen and noble gases such as for example argon. It is possible to conduct the heating and agitating under a nitrogen gas atmosphere. Processing under an inert atmosphere may be applied to reduce, preferably minimize and most preferably substantially exclude the presence of oxygen. In practice, the inert atmosphere may still contain minor amounts of oxygen. Typically, the inert atmosphere has an oxygen level of less than 90 g oxygen/m³ of the atmosphere, preferably less than 50 g/m³, more preferably less than 30 g/m³, even more preferably less than 25 g/m³ and most preferably less than 23 g/m³. Vacuum conditions that may be applied are, for example, subpressures within the range of from 70 to 1 kPa, preferably from 30 to 2 kPa, and most preferably from 10 to 2.5 kPa.

An example of a non-volatile oily active ingredient that may be incorporated in the oleogel composition of the present invention is simethicone, which is a silicone oil.

Silicone oils have been used in medicines, cosmetics, and medical devices for well over 60 years. Polydimethylsiloxane (PDMS) is commonly used in oral anti-flatulent remedies, and as the active pharmaceutical ingredient in many topically applied skin protectants. Dimethicone is chemically defined as a fully methylated siloxane polymer with trimethylsiloxy end block units. In North America, simethicone is a more common anti-flatulent remedy and is a mixture of dimethicone with four to seven weight percent silicone dioxide. Several United States Pharmacopoeia (USP) monographs exist that describe simethicone, as a raw material and finished dosage forms that utilize simethicone as the active pharmaceutical ingredient, including capsules, emulsions, and oral suspensions. Bulk simethicone that complies to the USP and European Pharmacopoeia monographs is produced by Dupont for the pharmaceutical market. Simethicone is available as the active pharmaceutical ingredient (API) in many antacid remedies, under a variety of brand names. Simethicone does not act to prevent gas formation in the gut, rather, it acts by decreasing the surface tension of gas bubbles in vivo, causing them to “flatten” and combine, thus allowing them to pass more easily.

In the present oleogel composition, simethicone may be present in an amount of 1%-36% by total weight. The ratio of simethicone to the second oil is preferably in the range of from 30:70 to 40:60.

Another example of a non-volatile oily active ingredient that may be incorporated in the oleogel of the present invention is cannabinoid (CBND in the following) oil.

CBNDs are chemical compounds produced by the plant Cannabis sativa. In recent years, CBNDs have been used to treat a number of medical conditions such as nausea and vomiting caused by anti-cancer medication, loss of appetite in people with AIDS, to alleviate neuropathic pain, spasticity, overactive bladder and other symptoms of multiple sclerosis, and to treat seizures associated with Lennox-Gastaut syndrome, Dravet syndrome and tuberous sclerosis complex. The CBNDs used for these purposes may be oil extracts of C. sativa comprising inter alia the active ingredients tetrahydrocannabinol or cannabidiol, or they may be synthetic versions of the active ingredients. The CBND oils may be administered as oil filled capsules, oral solutions or mouth sprays. Formulating CBND oils as ethylcellulose oleogel compositions according to the present invention may improve the convenience of taking the medication and may consequently improve patient compliance.

In the present oleogel composition, CBND oil may be present in an amount of 51%-85% by total weight. The ratio of CBND oil to the second oil is preferably in the range of from 65:35 to 50:50, preferably about 60:40.

### EXAMPLES

### Example 1: Ethylcellulose Oleogel Comprising Simethicone

### Oleogel Synthesis

Oleogels were made on an IKA hotplate using a silicone oil heating bath. To an 8 oz jar, the desired amount of simethicone and/or sunflower oil was weighed, and the two components were stirred together by hand to create a single phase. The ETHOCEL™ polymer was then weighed into a weigh boat and added to the jar. The powder was stirred into the oil by hand to create a slurry consistency. The jar was then clamped into the oil bath, stirred at 400-500 rpm with an overhead stirrer equipped with a cowls blade, and heated to 155-160° C. Once the temperature reached 155-160° C., the mixture was held for 60-70 minutes, watching for all the solid polymer to dissolve into the oil. Once the polymer was dissolved, the jar was removed from the heat and allowed to cool at room temperature to form the gel.

### Foaming Test

A mixture of Triton X-100 surfactant in water was used as the foaming solution. To 1 L of water, 10 g of Triton X-100 was added and stirred with an overhead stirrer for 30 min until all of the surfactant was incorporated.

For each foam test, 100 mL of the foam solution was added to an 8 oz. glass jar. To this, either 500 μL of a 1% simethicone solution (in DI water) or 15 mg of simethicone oleogel were added. These equated to the same simethicone dosage per jar. The jar was manually shaken for 10 seconds to create a foam layer, and then a stopwatch was used to time how long it took for the foam layer to flatten. Each experiment was repeated 3 times and the data averaged.

### Gel Formation

Several oleogel samples were made at different concentrations of simethicone. Initially, 100% simethicone was mixed with ˜17% ETHOCELM std. 20 (EC20) to create a gel. While heating to dissolve the ETHOCEL™, the mixture began to turn gray and small off-color specs were noticed in the jar. When the jar was taken off the heat and allowed to cool, it was apparent the ETHOCEL™ had not dissolved in the simethicone oil and had begun to degrade. It was determined that there was likely residual sulfuric acid in the simethicone, as sulfuric acid is used as a ring-opening catalyst during the synthesis of simethicone. The combination of residual acid and increased temperature was likely the cause of the ETHOCEL™ degradation.

To circumvent exposure to residual acid and mix the ETHOCEL™ with an oil that it would be soluble/miscible with, a mixture of simethicone and sunflower oil was used to make the next gels. Table 1 summarizes several oleogels made with varying ratios of sunflower oil and simethicone at 17% EC20 loading. As the amount of simethicone was reduced, the gel formation improved. Any amount of simethicone above 40% resulted in non-fully formed gels, gritty solutions, and/or separation of the simethicone and sunflower oil components. For example, when 70% simethicone and 30% sunflower oil was used, the result was a goopy solution with a clear simethicone layer and a pseudo gel of the ETHOCEL™ with the sunflower oil. However, when 40% simethicone and 60% sunflower oil was used, the ETHOCEL™ was able to gel with the sunflower oil and contain the simethicone in that structure, resulting in a friable, waxy, gel substance. Table 1 summarizes the gel formation experiments using different ratios of simethicone to sunflower oil.

TABLE 1

| Oleogel composition | Gel formation |
|---|---|
| Simethicone + EC20 | EC20 not dissolved in simethicone; no gel |
| (17% w/w) | formation |
| Simethicone:sunflower oil | Phase separation between EC20 + sunflower oil |
| 70:30 + EC20 (17% w/w) | and simethicone |
| Simethicone:sunflower oil | Separation of simethicone |
| 60:40 + EC20 (17% w/w) | |
| Simethicone:sunflower oil | Separation of simethicone |
| 50:50 + EC20 (17% w/w) | |
| Simethicone:sunflower oil | EC20 gels with sunflower oil, simethicone |
| 40:60 + EC20 (17% w/w) | contained in the gel structure. Friable waxy gel |

Once it was determined that a ratio of 40:60 simethicone:sunflower oil was the tipping point for gel formation, the impact of different viscosity grades of ETHOCEL™ was investigated. Table 2 is a summary of gels made at 40:60 and 60:40 ratios of simethicone to sunflower oil with 4 different grades of ETHOCEL™ (EC) at 17% total polymer loading. All formulations where a majority simethicone was used resulted in no gel formation and insolubilized ETHOCEL™ as expected from previous results. Gels made with a 40:60 blend of simethicone and sunflower oil made different quality of gels depending on the viscosity grade of ETHOCEL™ used. Gels that were made using the highest viscosity grade, and therefore the highest molecular weight, of ETHOCEL™, resulted in discoloration and no gel formation. As the molecular weight decreased, the gels become more solid and stable until the lowest molecular weight. The gel made with EC 7 had a more gel like consistency than a true solid, however it would hold its shape when small indents were made with a spatula. It was noted that after aging for two weeks, the simethicone began to separate from the EC 7 gel. Table 2 summarizes the gel formation experiments using 60:40 and 40:60 ratios of simethicone and ethylcellulose at four different viscosities.

TABLE 2

| Ratio | Ethylcellulose | |
|---|---|---|
| simethicone:sunflower | w/w | Gel formation |
| 60:40 | EC45 17% | Discoloured, no gel formed |
| 40:60 | EC45 17% | Discoloured, no gel formed |
| 60:40 | EC20 17% | No gel formed |
| 40:60 | EC20 17% | Solid, stable gel formed |
| 60:40 | EC10 17% | No gel formed |
| 40:60 | EC10 17% | Solid, stable gel formed |
| 60:40 | EC7 17% | No gel formed |
| 40:60 | EC7 17% | Soft gel formed, phase separation |
| | | after 2 weeks |
| 40:60 | EC20 9% | Flowable gel formed, phase |
| | | separation |

The discoloration and poor gel formation of the highest molecular weight samples of ETHOCEL™ support the hypothesis of acid induced degradation of ETHOCEL™ during the gel synthesis process. Higher molecular weight grades have larger polymer chains that could be more susceptible to degradation, which is why the degradation was only observed for EC 45. As the molecular weight of the polymer decreased, the degradation was not observed, because the starting material already had shorter polymer chains.

Having to dilute the simethicone with sunflower oil reduced the overall drug loading that could be achieved in the gels. With a 40:60 blend ratio of simethicone to sunflower oil, and 17% polymer content to achieve the gels shown previously, the total drug loading was 33%. A gel made with only 9% polymer content resulted in a drug loading of 36%. The resulting gel was flowable and showed some separation of the silicone oil from the remaining mass. A polymer level between 9 and 17% permits sufficient gelling with the sunflower oil to fully entrap the silicone oil within the gel structure.

### Application De-Foaming Results

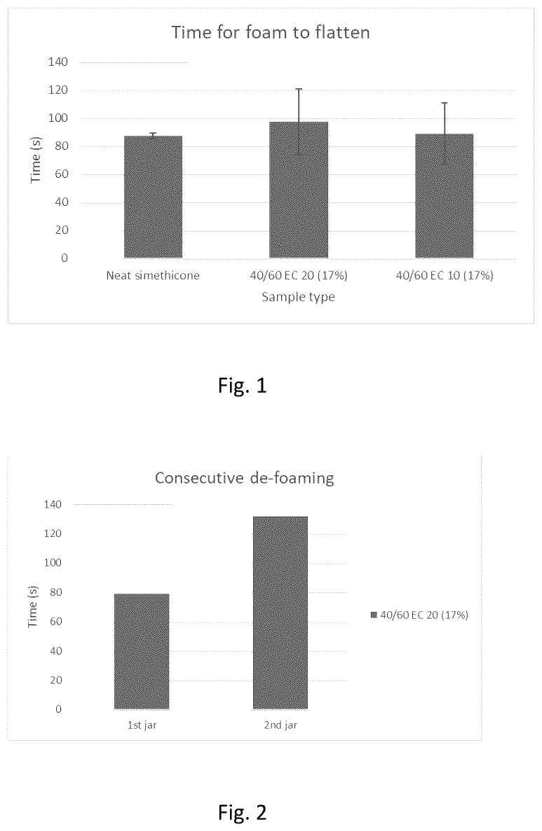

De-foaming tests were performed to demonstrate that the simethicone oleogel dosage form still remained active in the application. The tests were performed using the USP test as guidance. Some deviations occurred, mainly due to the lack of an automated wrist action shaker, so the solutions were foamed by manually shaking the jars. The results of the de-foaming test are shown in FIG. 1. The neat simethicone oil was able to completely flatten the foam within 85-90 seconds and had excellent reproducibility. The two gel samples were also able to flatten the foam in around 85-100 seconds, but the variability was higher. The increase in variability was due to the different morphology of the gel used. Since the gels are friable solids, the gel used in the de-foaming test could either be small discrete particles, or one larger gel mass, and the surface area was then very different. With the smaller particles, the de-foaming time was faster, while when it was a larger gel mass, de-foaming took longer. Changing the morphology could allow for an additional lever in modulating a desired release profile.

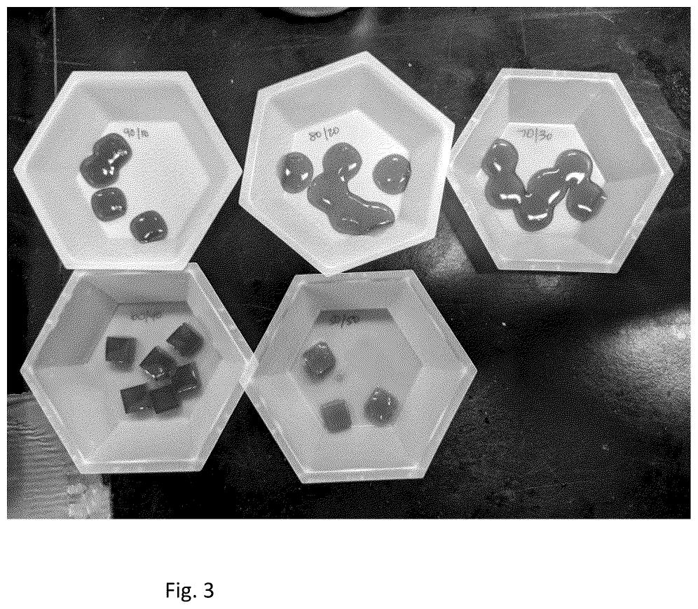

Since the oleogels are not water soluble, and the simethicone is entrapped within the gel matrix, theoretically the dosage form could continue to release active after being used initially. A gel sample was weighed out and placed in a mesh basket, which was topped with a rubber stopper to prevent the gel from leaving the basket. The whole basket was immersed in the foam solution and shaken. The time required for the foam to flatten was recorded, and the basket was immediately removed and placed into a new foam solution. The new foam solution was then shaken, and the time was recorded for that foam to flatten. FIG. 2 shows the time results from the sequential defoaming. The gel sample was capable of breaking up the foam in the second jar, although it may have taken longer to achieve the flattening. The data was limited to only one replicate to prove the concept.

### Conclusion

Successful simethicone oleogels retained their functionality in de-foaming tests. The simethicone loaded gels were able to flatten a foam solution equivalently to the neat simethicone oil. It was observed that the surface area of the simethicone oleogel was important for consistent results, as higher surface area led to faster release of the oil to the foam/water interface. A sequential de-foaming test was also performed to demonstrate a pseudo-controlled release effect of the simethicone. A simethicone oleogel was used to flatten a soap solution, and the remaining intact gel was transferred to a new soap solution and retained its ability to dissipate the foam.

### Example 2: Ethylcellulose Oleogel Comprising CBND Oil

### Oleogel Synthesis

Oleogels were made on an IKA hotplate using a silicone oil heating bath. CBND oil extract was pre-heated at 90° C. until a flowable liquid was obtained. To an 8 oz jar, the desired amount of pre-heated CBND oil extract and/or sesame oil was weighed, and the two components in the jar were clamped into the oil bath and stirred at 200 rpm with a cowls blade to form a single phase. The ETHOCEL™ polymer was then weighed into a weigh boat and slowly added to the stirring oil. Once the temperature reached 155-160° C., the mixture was held for 35-40 minutes, watching for all the solid polymer to dissolve into the oil. Once the polymer was dissolved, the molten oleogel was poured into molds and allowed to cool at room temperature to form the gel. The molded forms were placed in bowls and left at room temperature for one week. The stability (as shape retention) was determined visually as shown in the table below.

| Ratio | Ethylcellulose | |
|---|---|---|
| CBND:sesame | w/w | Gel formation |
| 100:0 | EC45 15% | Hard solid, molded forms do not retain |
| | | shape upon storage, tacky surface |
| 90:10 | EC45 15% | Hard solid, molded forms do not retain |
| | | shape upon storage, tacky surface |
| 80:20 | EC45 15% | Hard solid, molded forms do not retain |
| | | shape upon storage, slightly tacky |
| | | surface |
| 70:30 | EC45 15% | Hard solid, molded forms do not retain |
| | | shape upon storage |
| 60:40 | EC45 15% | Gel formation, molded forms retain |
| | | shape, no tackiness |
| 50:50 | EC45 15% | Gel formation, molded forms retain |
| | | shape, no tackiness, minimal sesame oil |
| | | weeping |

1. An oleogel composition comprising an ethylcellulose polymer, a first, non-volatile oil which is an active ingredient and a second oil selected from the group consisting of triglyceride oils and mineral oils.

2. An oleogel composition according to claim 1, wherein the ethylcellulose has a viscosity in the range of 5-60 mPa·s when determined as a 5% by weight solution in 80% toluene and 20% ethanol as solvent at 25° C. in an Ubbelohde viscometer.

3. An oleogel composition according to claim 1, wherein the ethylcellulose has a viscosity of 5-8 mPa·s when determined as a 5% by weight solution in 80% toluene and 20% ethanol as solvent at 25° C. in an Ubbelohde viscometer.

4. An oleogel composition according to claim 1, the ethylcellulose is present in an amount of 10-20% by total weight of the composition.

5. An oleogel composition according to claim 1, wherein the triglyceride oil is selected from the group consisting of sunflower oil, rapeseed oil, grape kernel oil, soybean oil, corn oil, olive oil, flaxseed oil, safflower oil, peanut oil, sesame oil, argan oil, rice bran oil, palm oil, cottonseed oil, linseed oil, almond oil, canola oil, or coconut oil.

6. An oleogel composition according to claim 1, wherein the first, non-volatile oil is simethicone.

7. An oleogel composition according to claim 6, wherein the simethicone is present in an amount of 1%-36% by total weight of the composition.

8. An oleogel composition according to claim 6, wherein the ratio of simethicone to the second oil is in the range of 30:70 to 40:60.

9. An oleogel according to claim 1, wherein the first, non-volatile oil is CBND oil.

10. An oleogel composition according to claim 9, wherein the CBND oil is present in an amount of 51-85% by total weight of the composition.

11. An oleogel composition according to claim 9, wherein the ratio of the CBND oil to the second oil is in the range of from 65:35 to 50:50.

12. An oleogel composition according to claim 1 further comprising a plasticizer.

13. An oleogel composition according to claim 12, wherein the plasticizer is stearic acid, triacetin, oleic acid, glyceryl monostearate, glycerol, propylene glycol or polyethylene glycol.

14. An oleogel composition according to claim 1 further comprising a surfactant.

15. An oleogel composition according to claim 14, wherein the surfactant is selected from the group of silicone surfactants or glyceryl monostearate.

16. A unit dosage form comprising a composition according to claim 1.

17. A unit dosage form which is a capsule comprising a composition according to claim 1.

18. A unit dosage form which is a molded dosage form comprising the composition according to claim 1.
